(19) United States
(12) Patent Application Publication
(10) Pub. No.: US 2016/0273031 A1
(43) Pub. Date: Sep. 22, 2016

(54) METHOD, KIT AND PREMIX FOR QUANTITATIVE AND DISCRIMINATIVE DETERMINATION OF NUCLEIC ACIDS, QDPCR; IN VITRO METHOD FOR THE QUANTITATIVE AND DISCRIMINATIVE DIAGNOSIS OF BIOLOGICAL ENTITIES

(71) Applicants: Spinomics Ltd. - EPP, Porto Alegre (BR); Neoprospecta Pesquisa e Consultoria S.A., Florianópolis (BR)

(72) Inventors: Rogério Margis, Porto Alegre (BR); Luiz Felipe Valter de Oliveira, Florianópolis (BR)

(73) Assignees: Spinomics Ltd. - EPP, Porto Alegre (BR); Neoprospecta Pesquisa e Consultoria S.A., Florianópolis (BR)

(21) Appl. No.: 15/037,123

(22) PCT Filed: Nov. 14, 2014
(86) PCT No.: PCT/BR2014/050013

§ 371 Date: May 17, 2016

(30) Foreign Application Priority Data
Nov. 18, 2013 (BR) BR102013029633-3

(51) Int. Cl. C12Q 1/68

(52) U.S. Cl. CPC C12Q 1/6851; C12Q 1/6818

(57) ABSTRACT

The present invention provides a process and a set of compounds of a kit for quantitative, specific and discriminative determination of nucleic acids (qdPCR). The products and processes of the invention correspond to a development which enables, at the same time and in a single closed system, to obtain a quantitative diagnosis of a gene locus, associated to a determinative and discriminative method between alleles or gene loci or even between orthologous genes of different biological entities present in samples/assays.

### FIELD OF THE INVENTION

The present invention refers to products and in vitro processes for diagnosis in molecular biology. More specifically, the present invention provides a process and a set of compounds of a kit for quantitative, specific and discriminative determination of nucleic acids. The products and processes of the invention correspond to a development which enables, at the same time and in a single closed system, to obtain a quantitative diagnosis of a gene locus, associated to a determinative and discriminative method between alleles and gene loci or even between orthologous genes from different biological entities present in samples/assays.

### BACKGROUND OF THE INVENTION

The advent of the polymerase chain reaction (PCR), in the 1980s, yielded to its inventor Kary Mullis the Nobel Prize and opened a new chapter in the history of Molecular Biology. This technology, which patent expired several years ago, allowed the amplification of nucleic acids (such as DNA) in virtually unlimited quantities, and its detection was greatly simplified. In the decades that followed intense, researches were made possible by this technology. In addition, several improvements derived from the PCR have been developed.

The U.S. Pat. No. 5,210,015, entitled “Homogeneous assay system using the nuclease activity of a nucleic acid polymerase” was filed on Aug. 6, 1990 by the company Hoffman-La Roche Inc and disclosed a process for the detection of a nucleic acid sequence. In said process, a marked oligonucleotide for amplification and nuclease action is used, which releases fragments of said marking which is detected when released. This technology is also already in public domain.

The U.S. Pat. No. 5,538,848, entitled “Method for detecting nucleic acid amplification using self-quenching fluorescence probe” was filed on Nov. 16, 1994 by the company Applied Biosystems and discloses a process for monitoring DNA amplification. In said process, it is also used the nuclease activity; one of the probes is marked with fluorescent substances, also using a quencher molecule.

The U.S. Pat. Nos. 5,210,015 and 5,538,848 enabled the real-time quantification of the amplification products from the measure of generated fluorescence, with the technique basis currently known as TaqMan®.

The U.S. Pat. No. 7,422,852, entitled “Detection of nucleic acid using linear beacons” was filed on Jun. 30, 2003 by the company Boston Probes Inc., and discloses another real-time quantification process of nucleic acid amplification products. Said process comprises the use of a beacon, consisting of a polymer with a region capable of giving energy and a region capable of receiving energy, such regions being separated by a certain length of nucleic acids. During the PCR amplification, signals are generated at each cycle, enabling another form of real time quantitation of nucleic acids.

The U.S. Pat. No. 7,387,887, entitled “Nucleic acid melting analysis with saturation dyes” was filed on Apr. 20, 2004 by University of Utah Research Foundation and Idaho Technology, Inc and discloses an approach of PCR including the use of a fluorescent intercalation which links in the region between the primers. As such fluorescent substances (as that known as SYBR® Green) are not linked to the primer, the amplification products can include nucleic acids containing polymorphisms, which are detected by high resolution melting analysis, or high resolution melting (HRM).

The US Patent Application 2004/0219565 A1, entitled “Oligonucleotides useful for detecting and analyzing nucleic acids”, discloses methods for obtaining profile of mRNAs and their splicing variants, mutants and alteration related to, for example, cancer. The method is related to in situ hybridization fluorescence, by LNA microarrays.

The US Patent Application WO 2011/032243 A1, entitled “Methods and kits for identification of animals with the greatest potential for desirable characteristics and for the early identification of deposition of fat in cattle”, discloses the identification, with molecular biology techniques, of markers for cattle fattening. In said document, several methods already known are disclosed, including PCR by the method TaqMan® or, alternatively, by High Resolution Melting, among others, but nothing like the present invention—since the use of both methodologies independently does not provide the advantages of the present invention.

From what is clear from the patent literature, there were not found documents that provide concurrently the benefits of TaqMan® strategy or other analogous strategies and the benefits of that known as HRM (high resolution melting) which provides discrimination between the variants of nucleotide sequence. Thus, there remains a need to develop a method or process that is practical, inexpensive and efficient, as well as being able to easily carry out proper quantitative and discriminative detection of nucleic acids concurrently and in a single analytical approach. In this context, and in a very interesting way, the great difficulty of choosing between one or other method has been a source of problems for the research and development of solutions in the industry. For example, very recently, the company Life Technologies released on its website (see http://find.lifetechnologies.com/qpcr/rapbattle/ytlink, accessed in Aug. 26, 2013) how difficult it has been for researchers to choose between one or other approach (TagMan® or SYBR®) indicating the apparent antagonism between them: “It's a dilemma that any real-time PCR researcher will most likely face in his or her career Do I choose TaqMan® or SYBR®? Inspired by the sentiments of researchers all over the world, Life Technologies has produced the “Rap Battle in the Lab” pitting TaqMan® and SYBR® chemistries against one another. Gather your lab mates and watch this epic confrontation unfold. Then, vote for a winner and submit your information for a chance to win an Apple® iPad mini or props featured in the music video.” The explicit mention that researchers from around the world have faced this problem until now—so as to prepare a publicity campaign comparing the processes which were considered incompatible so far with each other—reinforces the merits of the present invention: compatibilizing such methods and additionally providing advantages that none of them can provide individually, which is surprising to a person skilled in the art.

From what is clear from the investigated literature, there were not found documents anticipating or suggesting the teachings of the present invention, so that the solution proposed here, from the point of view of the inventors, has novelty and inventive activity against the prior art.

### SUMMARY OF THE INVENTION

The present invention provides a method compatibilizing the real-time quantitative PCR (as is the case of TaqMan®) and the method known as HRM (high resolution melting), which provides discrimination between nucleotide sequence variants. The present invention provides, at the same time and in a single closed system, obtaining a quantitative diagnosis of a gene locus, associated with a determinative and discrimination method between alleles or gene loci or even between orthologous genes from different biological entities present in samples/assays.

It is an object of the invention an in vitro process for the quantitative and discriminative determination of nucleic acids (qdPCR). In a preferred embodiment, the process for quantitative and discriminative determination of nucleic acids (qdPCR) of the invention comprises:
(i) a step of mixing a sample containing nucleic acid to be analyzed with a set of reagents comprising:
a pair of specific primers and one or more probes associated with fluorophore(s), as well as their respective quencher(s);
an intercalating fluorophore with distinct emission from that of fluorophore(s) associated with probe(s);
DNA polymerase, buffers, salts and dNTPs,
(ii) a step of amplification and cleavage of the fluorescent probe; and
(iii) a melting step.

It is another object of the invention a kit and a pre-mix for in vitro process for the quantitative and discriminative determination of nucleic acids. In an preferred embodiment, the kit and the pre-mix of the invention comprises:
a pair of specific primers and one or more probes associated with fluorophore(s), as well as their respective quencher(s);
a pair of specific primers and one or more probes associated with fluorophore(s), as well as their respective quencher(s); and
DNA polymerase, buffers, salts and dNTPs.

It is another object of the invention an in vitro process for quantitative and discriminative diagnosis of biological entities.

These and other objects invention will be immediately appreciated by person skilled in the art and by companies with interests in the sector, and will be described in details sufficient for their reproduction in the following description.

### BRIEF DESCRIPTION OF THE DRAWINGS

FIG. 1 (prior art) shows a schematic representation of a detection method of DNA or cDNA specific sequence by probe methodology associated with nuclease activity (TaqMan®). In A) the steps 1-5 are shown. In the first step, it is represented a double-stranded DNA molecule or cDNAs. In the second step, of annealing, a pair of specific primers and a fluorescent probe, which specifically anneals in the target sequence. In the third step, of elongation or polymerization, the double strands which are complementary to the mold and to the probe cleavage are produced by DNA polymerase, with fluorescence emission, which is proportional to the amount of cleaved probe. In the fourth step, it is represented the total of detectable fluorophore, released from probe, and the specific product amplified by the pair of primers with non-fluorescent nature and not detectable by the method (indicated by 5). In B) it is shown a schematic representation of the PCR amplification curve wherein the number of cycles is represented on the abscissa and the measured fluorescence or the fluorescence in log scale is represented on the ordinate.

FIG. 2 (prior art) shows a schematic representation of a method for detecting DNA or cDNA specific sequence by the methodology of intercalating fluorophore with HRM dissociation curve (SYBR® Method or equivalent). In A) the steps 1-5 are shown. In the first step, it is represented a double-stranded DNA molecule or cDNAs. In the second step, of annealing, a pair of primers specifically anneals in the target sequence. In the third step, of elongation or polymerization, the double strands which are complementary to the mold, with intercalation of the fluorophore and increase of the fluorescence emission intensity, proportional to the amount of amplified double strands. In the fourth step, it is represented the total of amplified specific products with intercalation of the fluorophore (indicated by 5). In B) it is shown a schematic representation of the derivative of the dissociation curve or melting and the normalized melting curve.

FIG. 3 shows a schematic representation of a preferred embodiment of the quantitative and discriminative process of the present invention (qdPCR). Innovative method associating the methods for detection of DNA or cDNA specific sequence by methodology of probe cleavage through nuclease activity (TaqMan®) and detection of amplified products and melting by intercalating fluorophore (Eva green, Syto® 9 or Syto® 13 or equivalent). It is indicated in A): 1) In the first step, it is represented a double-stranded DNA molecule or cDNAs. The denaturation step, with separation of the double-stranded DNA; 2) In the second step, of annealing, a pair of specific primers and a fluorescent probe, that specifically anneal in the target sequence. The annealing step, in which a pair of primers, an intercalating fluorophore and a probe (“s”) with fluorophore are used; 3) In the third step, of elongation and polymerization, the double strands which complementary to the mold and the probe cleavage are produced by DNA polymerase, with fluorescence emission, proportional to the amount of cleaved probe and also the intercalation of the fluorophore, producing amplified products with associated and differentiated fluorescence, followed by Step 4). In the fourth step, it is represented: the total of detectable fluorophore, released from probe (detection channel different than “1”), and the specific product amplified by the pair of primers with non-fluorescent nature and detectable by intercalating fluorophore, in the channel “1” of the real-time PCR equipment. In the fourth step, it is represented the total of specific products amplified with intercalation of fluorophore. In 5), it is shown the other fluorescence proportional to the amount of cleaved probes. In B), it is shown schematic representations of the PCR amplification curve wherein the number of cycles is represented on the abscissa and the fluorescence logarithm measured by probe cleavage is represented on the ordinate and detected in channel different than “1” in the real-time PCR apparatus. In C) it is shown schematic representations of the normalized melting curve, obtained in channel “1” of the real-time PCR apparatus, due to the intercalation of the fluorophore, for two samples containing polymorphic sequences.

FIG. 4 shows the results of qdPCR for the example 2, for six samples of tobacco, the normalized melting curves being shown, obtained in channel “1” of the real-time PCR apparatus, due to intercalation of fluorophore, for two samples containing polymorphic sequences. In A) NF represents a normalized fluorescence and in B) NF represents a normalized fluorescence minus the reference; T represents the temperature in Celsius degrees for both cases.

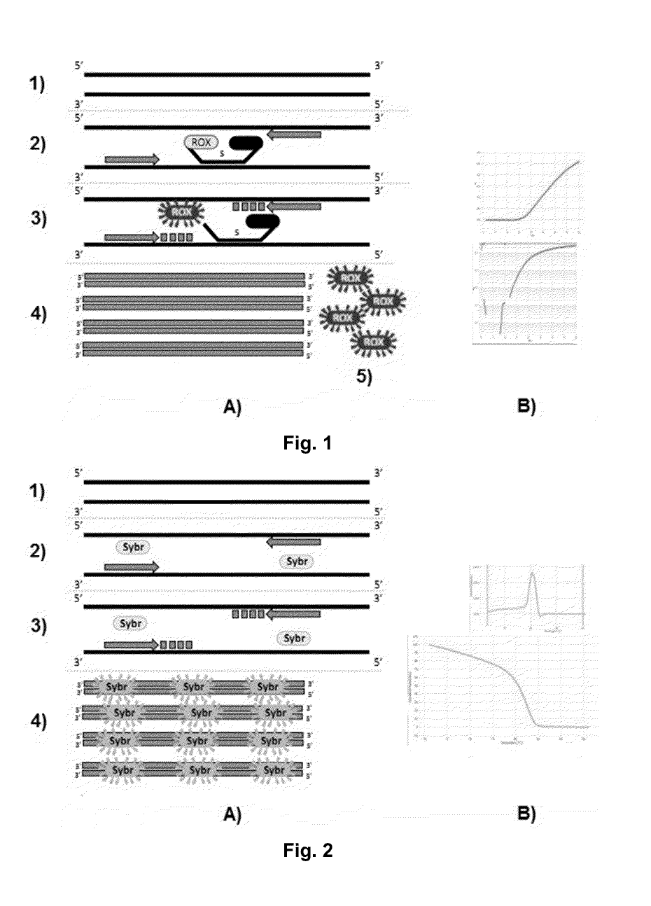

### DETAILED DESCRIPTION OF THE INVENTION

The process of the invention enables the compatibilization of the methods for detection of DNA or cDNA specific sequence by the methodology of probe cleavage through nuclease activity (for example, TaqMan®) and detection of amplified products and high resolution melting by intercalating fluorophore (for example, Syto® 9 or equivalent). The understanding of the difficulties of the prior art, overcome by the present invention, requires detailing about the particulars of the methods used until now, as well as its limitations.

In the methods based on degradation of nucleotide probes by exonuclease activity, including those known as TaqMan®, the high specificity is obtained by the combined use of specific fluorescent probes flanked by a pair of primers (or primers) also specific. Such pair of primers allows the specific amplification of the locus of interest. Fluorescent probes that hybridize within the amplified sequence further increases specificity and provide detection by fluorescence. The specificity is so great that this methodology is used for the discrimination between alleles, since changes in only one nucleotide can be detected for allelic discrimination. In this case, probes with different sequences and different fluorescence are used to discriminate one allele from another. This specificity is desirable, however limiting upon the existence of unknown nucleotide variations, such as SNPs, which may result in false negatives by no hybridization of the probe. In cases of possible existence of SNPs out of the probe annealing region, the method TaqMane will provide the amplification and fluorescence, however without detecting the existence of such variants—far less discrimination among them.

On the other hand, the high resolution melting method (HRM) allow the detection of unknown mutations, such as SNPs, in an amplified sequence—independently of the position taking place. But, such method does not provide the same specificity of amplification and quantification than TaqMan®. The HRM method is, however, more discriminative and potentially a detector of mutations.

However, one of the fluorophores (FAM) traditionally more used and coupled to one of the probes used in the methodology of TaqMan® for allelic discrimination has its maximum fluorescence in the same region of intercalating fluorophores commonly used in HRM (SYBR® family—see tables 1 and 2). In other words, the mere combination of the two methods, in the way known today, does not work, because, with the use of these traditional fluorophores, the user would be unable to differentiate the origin of each signal. Therefore, one of the additional elements of the present invention is the proper selection of the intercalating fluorophores (HRM) and those used for marking of the probes (TaqMan®), so they do not produce signal in the same fluorescence emission region. Thus, methods are compatibilized. For ready reference, some combinations of fluorophores are listed below, used for marking the probes (TaqMan®) or as intercalatings (HRM). The tables 1 and 2 below aim to facilitate and choose fluorophores by a person skilled in the art that wants to reproduce the present invention.

TABLE 1
Examples of fluorophores which can be used in HRM
and fitting according to wavelength (nm) of fluorescence
emission detection (filters 1-4).

| | | | |
|---|---|---|---|
| 1 (510) | 2 (540) | 3 (600) | 4 (674) |

| | | |
|---|---|---|
| SyBr ® Green | Syto ® 82 | Syto ® 64 |
| Eva green | | |
| Syto ® 9 | | |
| Syto ® 13 | | |

TABLE 2
Examples of fluorophores which can be used in TaqMan ® and
fitting according to wavelength (nm) of fluorescence
emission detection (filters 1-4).

| 1 (510) | 2 (540) | 3 (600) | 4 (674) |
|---|---|---|---|
| FAM | HEX | ROX | Cy5 |
| | JOE | Texas Red | |
| | Cy3 | | |

With proper choice of fluorophores, in the present invention, the following advantages are provided, at the same time: the quantitative detection as well as the high specificity (that come from real-time quantitative PCR with nuclease activity, such as TaqMan®), and the identification of nucleotide variants and their discrimination (that come from HRM). Hence, the use of the present invention allows not only the detection of alleles of interest (by processes as TaqMan®) but also unknowns allelic variants (by HRM). Thus, the process for quantitative and discriminative determination of nucleic acids (qdPCR) of the invention comprises:
(i) a step of mixing a sample containing nucleic acid to be analyzed with a set of reagents comprising:
a pair of specific primers and one or more probes associated with fluorophore(s), as well as their respective quencher(s);
an intercalating fluorophore with distinct emission from that of fluorophore(s) associated with probe(s);—DNA polymerase, buffers, salts and dNTPs,
(ii) a step of amplification and cleavage of the fluorescent probe; and
(iii) a melting step.

The person skilled in the art will know immediately what are the salts and buffers commonly used in reactions of this type.

### EXAMPLE 1

### qdPCR

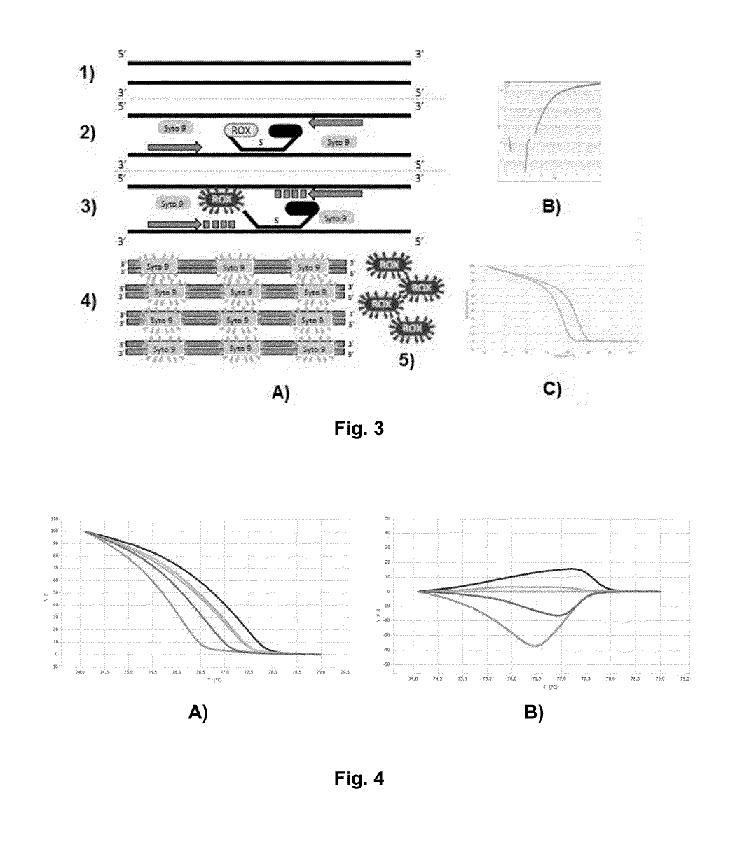

In a preferred embodiment, illustrated in FIG. 3, the process of the invention enables compatibilization of the detection methods of the DNA or cDNA specific sequence by methodology of probe cleavage through nuclease activity (for example, TaqMan®) and detection of amplified products and melting by intercalating fluorophore (Eva green, Syto® 9 or Syto® 13 or equivalent). It is indicated in A): 1) In the first step, it is represented a double-stranded DNA molecule or cDNAs. The step of denaturation, with separation of the double-stranded DNA; 2) In the second step, of annealing, a pair of specific primers and a fluorescent probe, that specifically anneal in the target sequence. The step of annealing, in which a pair of primers, an intercalating fluorophore and a probe (“s”) with fluorophore are used; 3) In the third step, of elongation or polymerization, the double strands which are complementary to the mold and the probe cleavage are produced by DNA polymerase, with fluorescence emission, proportional to the amount of cleaved probe and also the intercalation of the fluorophore, producing amplified products with associated and differentiated fluorescence, followed by Step 4). In the fourth step, it is represented: the total of detectable fluorophore, released from probe (detection channel different than “1”), and the specific product amplified by the pair of primers with non-fluorescent nature and detectable by intercalating fluorophore, in the channel “1” of the real-time PCR equipment. In the fourth step, it is represented the total of specific products amplified with intercalation of fluorophore. In 5) it is shown the other fluorescence proportional to the amount of cleaved probes. In B) it is shown schematic representations of the PCR amplification curve wherein the number of cycles is represented on the abscissa and the fluorescence logarithm measured by probe cleavage is represented on the ordinate and detected in channel different from “1” in the real-time PCR apparatus. In C) it is shown schematic representations of the normalized melting curve, obtained in channel “1” of the real-time PCR apparatus, due to the intercalation of the fluorophore, for two samples containing polymorphic sequences.

### EXAMPLE 2

### Kit and Pre-mix

The kit and the pre-mix of the invention comprises:
a pair of specific primers and one or more probes associated with fluorophore(s), as well as their respective quencher(s);
an intercalating fluorophore with distinct emission from that of fluorophore(s) associated with probe(s); and
DNA polymerase, buffers, salts and dNTPs.

In this preferred embodiment, whose results are illustrated in FIG. 4, the pre-mixes of qdPCR are placed in a single tube containing, in addition to all reagents necessary for the amplification reaction:
(i) a pair of universal primers for amplification of psbA gene of target plants of interest;
(ii) a specific probe for detecting the psbA chloroplast gene of tobacco (Nicotiana benthamiana) coupled to HEX fluorophore and blocker (Quencher) to provide the quantification of the expression of respective gene or detection of carrying organism thereof. The fluorophore emits in wavelength which is different (Filter 2 of apparatus) from the intercalating fluorophore; and
(iii) intercalating fluorophore (Syto® 13) to perform the HRM at the end of 40 cycles of qdPCR. For HRM, it is used the filter 1 of equipment.

With this approach of pre-mix for the amplification and HRM processes with qdPCR, in a single tube, the following advantages/features are provided:
(i) detection and/or quantification of a target gene of interest or its carrier organism;
(ii) the identification, through HRM, of polymorphisms in the amplified region; and
(iii) even in cases which would be negatives with the exclusive use of methodology TaqMan®, the approach of the invention provide the detection of amplification products, indicative fact of the existence of polymorphism(s) in the region annealed by the probe.

The approach of the invention provides concurrently and in a single reaction of qdPCR:
(i) the detection of polymorphisms and their discrimination; and
(ii) the high sensitivity in the amplification, further avoiding the occurrence of false negative cases.

### EXAMPLE 3

### qdPCR for Food Quality Control

The in vitro process for quantitative and discriminative diagnosis of biological entities of the present invention provides, among other applications, an improved food quality control. As an example, sausages are usually made with mixtures of meat of different animal species, such as bovine and swine. Thus, in this preferred embodiment, samples of sausages from different origins are submitted to qdPCR, aiming to identify and quantify the animal origin and ratio of these ingredients in the sausage. In preferred embodiment, pre-mixes of qdPCR are placed in a single tube containing, in addition to all reagents necessary for the amplification reaction:
(i) a pair of universal primers for amplification of the mitochondrial gene Cox1;
(ii) specific probes for detecting the bovine and swine gene Cox1 coupled to fluorophore HEX and ROX, respectively, and its blockers (Quencher) to provide the quantification of the expression of respective gene or detection of the originating organism thereof. The fluorophores emit in wavelengths different (Filters 2 and 3 of apparatus) from that of the intercalating fluorophore; and
(iii) intercalating fluorophore (Syto® 13) to perform HRM at the end of 40 cycles of qdPCR. For HRM, it is used the filter 1 of the equipment.

With this approach of qdPCR, in a single tube, the following advantages/features are provided:
(i) detection and/or quantification of bovine and/or swine gene Cox1 in the sausage sample;
(ii) the identification, through HRM, of any polymorphisms relating to a third source or other animal sources present(s) in the sausage; and
(iii) even in cases which would be negatives with the exclusive use of methodology TaqMan®, the approach of the invention provide the detection of amplification products, indicative fact of the existence of other animal sources present in the sausage.

Those skilled in the art will value the knowledge presented herein and may reproduce the invention in the shown embodiments and in other variants, encompassed within scope of the appended claims.

1. Process for quantitative and discriminative determination of nucleic acids (qdPCR), characterized by comprising:
(i) a step of mixing a sample containing nucleic acid to be analyzed with a set of reagents comprising:
a pair of specific primers and one or more probes associated with fluorophore(s), as well as their respective quencher(s);
an intercalating fluorophore with distinct emission from that of fluorophore(s) associated with probe(s);
DNA polymerase, buffers, salts and dNTPs,
(ii) a step of amplification and cleavage of fluorescent probe; and
(iii) a melting step.

2. Process, according to claim 1, characterized in that the fluorophore associated with probe is selected among HEX and/or ROX and the intercalating fluorophore is Syto® 13.

3. Kit for quantitative and discriminative determination of nucleic acids (qdPCR) characterized by comprising:
a pair of specific primers and one or more probes associated with fluorophore(s), as well as their respective quencher(s);
an intercalating fluorophore with distinct emission from that of fluorophore(s) associated with probe(s); and
DNA polymerase, buffers, salts and dNTPs.

4. Pre-mix for quantitative and discriminative determination de nucleic acids (qdPCR) characterized by comprising:
a pair of specific primers and one or more probes associated with fluorophore(s), as well as their respective quencher(s);
an intercalating fluorophore with distinct emission from that of fluorophore(s) associated with probe(s); and
DNA polymerase, buffers, salts and dNTPs.

5. In vitro process for quantitative and discriminative diagnosis of biological entities characterized by comprising:
(i) a step of mixing a sample containing nucleic acid to be analyzed with a set of reagents comprising:
a pair of specific primers and one or more probes associated with fluorophore(s), as well as their respective quencher(s);
an intercalating fluorophore with distinct emission from that of fluorophore(s) associated with probe(s);
DNA polymerase, buffers, salts and dNTPs,
(ii) a step of amplification and cleavage of the fluorescent probe; and
(iii) a melting step.
